(19) United States
(12) Patent Application Publication
(10) Pub. No.: US 2023/0070682 A1
(43) Pub. Date: Mar. 9, 2023

(54) TUBULAR STRUCTURE LIGATOR

(71) Applicant: Vivek Harihar, Shimago (IN)

(72) Inventor: Vivek Harihar, Shimago (IN)

(21) Appl. No.: 17/760,221

(22) PCT Filed: Mar. 25, 2020
(86) PCT No.: PCT/IB2020/052796

§ 371 Date: Aug. 5, 2022

(30) Foreign Application Priority Data
Feb. 7, 2020 (IN) 202041005611

(51) Int. Cl. A61B 17/128

(52) U.S. Cl. CPC A61B 17/128; A61B 2017/00407

(57) ABSTRACT

A surgical instrument is disclosed. The instrument includes a handle configured to move between an open position and a closed position; at least one clamping structure coupled to the handle. The at least one clamping structure includes a first set of pushing members operatively coupled to the handle. The movement of the handle from open position to the closed position facilitates the movement of pushing members into longitudinal direction. The at least one clamping structure includes a set of constrictors coupled to the set of the pushing members such that movement of the set of the pushing members enables movement of the set of constrictors from a designed position in which the set of constrictors are positioned away from each other and to a contracted position in which the set of constrictors are positioned closed to each other.

### TECHNICAL FIELD

The present disclosure relates to surgical instruments. More particularly, the present disclosure is related to surgical instruments that clamp and cut tubular body tissue or structure simultaneously.

### BACKGROUND

The background description includes information that may be useful in understanding the present invention. It is not an admission that any of the information provided herein is prior art or relevant to the presently claimed invention, or that any publication specifically or implicitly referenced is prior art.

Surgical instruments are typically known in a wide range of embodiments as scissors and clips, or the like. In surgery, surgical clips, in particular, in the form of so-called ligature clips, are increasingly being used for hemostasis of blood vessels and for clamping and occluding other hollow organs. So-called double-shank clips, for example, are also used. Such type of clips comprises clamping arms with free ends which are connected in pairs. With such double-shank clips, which may be formed from a wire ring closed within itself, vessels or hollow organs can even be easily occluded at two locations separate from each other. However, application of the clips requires meticulous dissection of tissue, during which, the adjacent vital structures may get damaged.

In addition, the clips are used in combination with another instrument called applicator. However, with the application of such combination, the clips dislodge itself from the applicator into an abdominal cavity even before applying. The slippage and dislodgement of clip in the abdominal cavity may cause haemorrhage, stump leak, clip cholelithiasis, abscess formation, bowel perforation etc., after the surgery.

Therefore, there is a need of an improved surgical instrument that can overcome above-mentioned challenges in the art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### OBJECTS OF THE INVENTION

A general object of the present disclosure is to provide an improved surgical instrument that is capable of performing operation of clip applicator as well as scissor.

Another object of the present disclosure is to provide an surgical instrument that reduces total time spent in the surgery.

Another object of the present disclosure is to provide an surgical instrument that can be used in verities of operations (such as Appendectomy, Cholecystectomy, Splenectomy, Bowel resection, Renal transplant, Female sterilization, Varicose Vein operation, Thyroidectomy, for taking venous graft in Cardiac bypass and other various surgeries) in different fields of surgery like General surgery, Gynaecological surgeries, Gastro-intestinal surgeries, Urology, Vascular surgeries and cardiac surgeries.

Another object of the present disclosure is to provide a tubular structure ligator that ligates appendix and appendicular artery together, so that this 30 minutes procedure can be done within 2-4 minutes practically.

Another object of the present disclosure is to provide a surgical instrument that can be easily operated because of its compact size in minimal access surgeries like laparoscopic and robotic surgeries, where there will be limited operating space.

Another object of the present disclosure is to provide an surgical instrument that can be easily operated without damaging or disturbing the adjacent structures.

Another object of the present disclosure is to provide an surgical instrument that minimizes the need of tissue dissection.

These and other objects of the present invention will become readily apparent from the following detailed description taken in conjunction with the accompanying drawings.

### SUMMARY

The present disclosure relates to surgical instruments. More particularly, the present disclosure is related to surgical instruments that clamp and cut tissue simultaneously.

In an aspect of the present disclosure provides a surgical instrument, the instrument comprising: a handle configured to move between an open position and a closed position; at least one clamping structure coupled to the handle, the at least one clamping structure comprising: a first set of pushing members operatively coupled to the handle, wherein movement of the handle from open position to the closed position facilitates the movement of pushing members into longitudinal direction; a set of constrictors coupled to the set of the pushing members such that movement of the set of the pushing members enables movement of the set of constrictors from a designed position in which the set of constrictors are positioned away from each other and to a contracted position in which the set of constrictors are positioned closed to each other, wherein movement of the set of constrictors into contracted position facilitate clamping of a tissue.

In an embodiment, the instrument comprises a cutting structure that comprises: a trigger configured with the handle and to switch between an extended position and a pushed position; a pushing member configured with the trigger such that switching of the trigger from the extended position to the pushed position enables linear movement of the pushing member; and a cutting tool coupled with the pushing member such that the movement of the pushing member moves the cutting tool to cut the tissue.

In an embodiment, the cutting structure comprises two rotating members, the two rotating members being movably configured on opposite side of the pushing member in longitudinal direction, wherein one of the two rotating members is movably coupled to the trigger such that switching of the trigger from the extended position to the pushed position facilitates rotation of the one of the two rotating members, and wherein another one of the two rotating members is movably coupled to the cutting tool such that rotation of the another one of the two rotating members facilitates operation of the cutting tool.

In an embodiment, the at least one clamping structure comprises: a second set of pushing members coupled to the handle; and at least one winding member movably coupled to the second set of pushing members such that movement of the second set of pushing members enables the movement of the at least one winding member, wherein the set of constrictors are positioned with a predefined gap between adjacent edges of the corresponding constrictors, and wherein the at least winding member is configured to pass through the gap between two adjacent constrictors.

In an embodiment, the instrument comprises a set of tubular members having hollow structure, each of the tubular members comprising each of the first and second sets of the pushing members.

In an embodiment, each of the tubular members comprises a bob coupled to the pushing member of the corresponding tubular member.

In an embodiment, the at least one clamping structure comprises a first clamping structure and a second clamping structure, wherein the first clamping structure is arranged with the second clamping structure, and wherein the cutting structure is configured between the first clamping structure and the second clamping structure.

In an embodiment, the surgical instrument comprises a first lever coupled with the first clamping structure and a second lever coupled with the second clamping structure.

In an embodiment, the set of the constrictors are positioned in a curved shape.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates exemplary perspective view of a proposed surgical instrument, in accordance with an embodiment of the present disclosure.

FIG. 2 illustrates exemplary perspective views of a proposed surgical instrument with at least one clamping structure, in accordance with an embodiment of the present disclosure.

FIG. 3 illustrates exemplary perspective views of a proposed surgical instrument with a cutting structure, in accordance with an embodiment of the present disclosure.

FIGS. 4A-4C illustrate exemplary perspective representation of movement of the winding member, in accordance with an embodiment of the present disclosure.

FIGS. 5A-5D illustrate exemplary perspective representation of cutting operation of the proposed surgical instrument, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

In the following description, numerous specific details are set forth in order to provide a thorough understanding of embodiments of the present invention. It will be apparent to one skilled in the art that embodiments of the present invention may be practiced without some of these specific details.

If the specification states a component or feature “may”, “can”, “could”, or “might” be included or have a characteristic, that particular component or feature is not required to be included or have the characteristic.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Exemplary embodiments will now be described more fully hereinafter with reference to the accompanying drawings, in which exemplary embodiments are shown. This disclosure may however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Various terms are used herein. To the extent a term used in a claim is not defined, it should be given the broadest definition persons in the pertinent art have given that term as reflected in printed publications and issued patents at the time of filing.

Embodiments explained herein relates to surgical instruments. More particularly, the present disclosure is related to surgical instruments that clamp and cut tissue simultaneously.

In an aspect of the present disclosure provides a surgical instrument. The instrument may include a handle configured to move between an open position and a closed position; at least one clamping structure coupled to the handle. The at least one clamping structure may include a first set of pushing members operatively coupled to the handle. The movement of the handle from open position to the closed position may facilitate the movement of pushing members into longitudinal direction. The at least one clamping structure may include a set of constrictors coupled to the set of the pushing members such that movement of the set of the pushing members may enable movement of the set of constrictors from a designed position in which the set of constrictors are positioned away from each other and to a contracted position in which the set of constrictors are positioned closed to each other. The movement of the set of constrictors into contracted position may facilitate clamping of a tissue.

In an embodiment, the instrument may include a cutting structure that may include: a trigger configured with the handle and to switch between an extended position and a pushed position; a pushing member configured with the trigger such that switching of the trigger from the extended position to the pushed position enables linear movement of the pushing member; and a cutting tool coupled with the pushing member such that the movement of the pushing member may move the cutting tool to cut the tissue.

In an embodiment, the cutting structure may include two rotating members. The two rotating members may be movably configured on opposite side of the pushing member in longitudinal direction. The one of the two rotating members may be movably coupled to the trigger such that switching of the trigger from the extended position to the pushed position may facilitate rotation of the one of the two rotating members. Another one of the two rotating members is movably coupled to the cutting tool such that rotation of the another one of the two rotating members may facilitate operation of the cutting tool.

In an embodiment, the at least one clamping structure may include asecond set of pushing members coupled to the handle; and at least one winding member movably coupled to the second set of pushing members such that movement of the second set of pushing members enables the movement of the at least one winding member. The set of constrictors may be positioned with a predefined gap between adjacent edges of the corresponding constrictors. The at least winding member may be configured to pass through the gap between two adjacent constrictors in a concentric circular manner.

In an embodiment, the instrument may include a set of tubular members having hollow structure. Each of the tubular members may include each of the first and second sets of the pushing members.

In an embodiment, each of the tubular members may include a bob coupled to the pushing member of the corresponding tubular member.

In an embodiment, the at least one clamping structure may include a first clamping structure and a second clamping structure. The first clamping structure may be arranged with the second clamping structure. The cutting structure may be configured between the first clamping structure and the second clamping structure.

In an embodiment, the surgical instrument may include a first lever coupled with the first clamping structure and a second lever coupled with the second clamping structure.

In an embodiment, the set of the constrictors may be positioned in a curved shape.

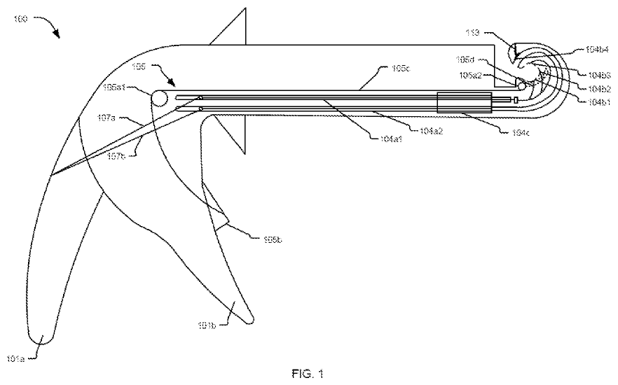

FIG. 1 illustrates exemplary perspective view of a proposed surgical instrument 100, in accordance with an embodiment of the present disclosure. As illustrated in FIG. 1, the proposed surgical instrument 100 (interchangeably referred to as instrument 100) may include a handle 101 configured to move between an opened position and a closed position. The instrument 100 may include at least one clamping structure 104 coupled to the handle 101. In another exemplary embodiment, the handle 101 may include a movable member 101a and a fixed member 101b. Alternatively, the handle 101 may include a fixed member 101a and a movable member 101b. The open position may correspond to a position in which the fixed member and the movable member may be kept at predefined distance. The closed position may correspond to a position in which the movable member is moved closer to the fixed member. The fixed member and the movable member may be pivotally fixed to a particular point. In order to bring the handle from open position to the closed position, movable member may be rotated about the pivot point towards the fixed member.

In an embodiment, the at least one clamping structure 104 (interchangeably referred to as clamping structure 104) may include a first set of pushing members 104a1 operatively coupled to the handle 101. The first set of pushing members 104a1 may be movably coupled to the handle 101 such that the movement of the handle 101 from open position to the closed position may enable the movement of the first set of pushing members 104a1 into longitudinal direction (forward direction). In another embodiment, movement of the handle 101 from closed position to the opened position may enable the movement of the first set of pushing members 104a1 into longitudinal direction (backward direction).

In another embodiment, the instrument may include a lever 107 coupled to the handle 101 at one end. The other end of the lever 107a may be coupled to the first set of the pushing members 104a1. However, it would be appreciated by a person skilled in the art that for multiple pushing members, multiple levers may also be configured. Configuration of the lever 107 with the first set of pushing members 104a1 and the handle 101 may be such that the movement of the handle 101 may enable the movement of the lever 107a that may facilitate the movement of the first set of pushing members 104a1.

In another embodiment, the instrument 100 may also include a set of tubular members having hollow structure. Each of the set of tubular members may include the first set of the pushing members 104a1. In another embodiment, each of the tubular members may include a bob coupled to the pushing member of the corresponding tubular member.

In an embodiment, the clamping structure 104 may include a set of constrictors 104b1, 104b2, 104b3 (collectively termed as constrictors 104b) movably coupled to the first set of the pushing members 104a1. The coupling of the first set of the pushing members 104a1 may be such that movement of the first set of the pushing members 104a1 enables movement of the constrictors 104b from a designed position in which the constrictors 104b are positioned away from each other and to a contracted position in which the constrictors 104b are positioned closed to each other. The movement of the set of constrictors into contracted position may facilitate clamping of a tissue.

As shown in FIG. 1, the constrictors 104b may be positioned in a curved shape. In an exemplary embodiment, the constrictors 104b may be arranged in a semi-circular shape to form a hook-like structure.

In an embodiment, the clamping structure 104 may include a second set of pushing members 104a2 that may be coupled to the handle 101. The clamping structure 104 may include at least one winding member 104c movably coupled to the second set of pushing members 104a2. The coupling between the winding member 104c and the second set of pushing members 104a2 may be such that movement of the second set of pushing members 104a2 may enable the movement of the at least one winding member 104c. In an embodiment, each of the first and the second set of pushing members may include one or more pushing members. However, a number of the pushing members in the first set of pushing members may be same as the number of the pushing members in the second set of pushing members.

In an embodiment, the set of constrictors 104b may be positioned with a predefined gap between adjacent edges of the corresponding constrictors 104b. The predefined gap may allow the at least winding member 104c to pass through the gap between two adjacent constrictors. In an exemplary embodiment, the at least winding member 104c may be wound around the tissue (such as appendix) in circular fashion after passing through the opening.

In an embodiment, configuration of lever and tubular member may be also applicable for the second set of pushing members, similar to the first set of pushing members.

In an embodiment, the at least one clamping structure 104 may include a first clamping structure 1041 and a second clamping structure 1042. In this case, the first clamping structure 1041 may be coupled to a first set of levers 107a and 107b and the second clamping structure may be coupled to a second set of levers (not shown). Each of the first and the second clamping structures may include the first set of pushing members and the second set of pushing members. In an embodiment, the first clamping structure may be arranged adjacently to the second clamping structure.

In an embodiment, as shown in FIG. 1, levers 107a and 107b may be configured to coupled the first set of pushing members 104a1 and the second set of pushing members 104b2, respectively.

In an embodiment, the instrument 100 may include a cutting structure 105 that may include a trigger 105b configured with the handle and to switch between an extended position and a pushed position. In another embodiment, the cutting structure 105 may include a pushing member 105c configured with the trigger 105b. In an embodiment, switching of the trigger 105b from the extended position to the pushed position may enable linear movement of the pushing member 105c.

In an embodiment the cutting structure 105 may include a cutting tool 105d coupled with the pushing member 105c such that the movement of the pushing member 105c may move the cutting tool 105d to cut the tissue. In an embodiment, the cutting tool 105d may have knife like structure.

In an embodiment, the cutting structure 105 may be configured with the at least one clamping structure 104, In another embodiment, the cutting structure 105 may be configured between the first clamping structure 1041 and the second clamping structure 1042.

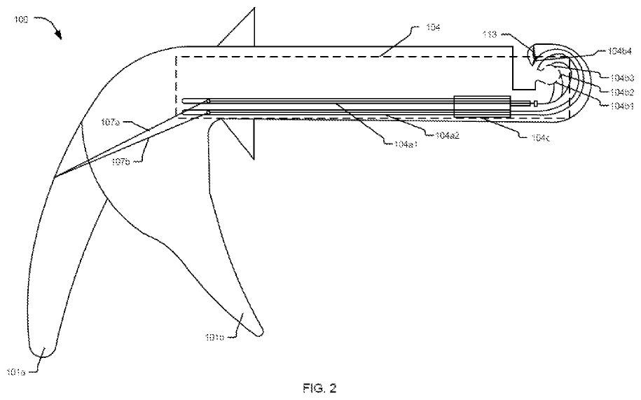

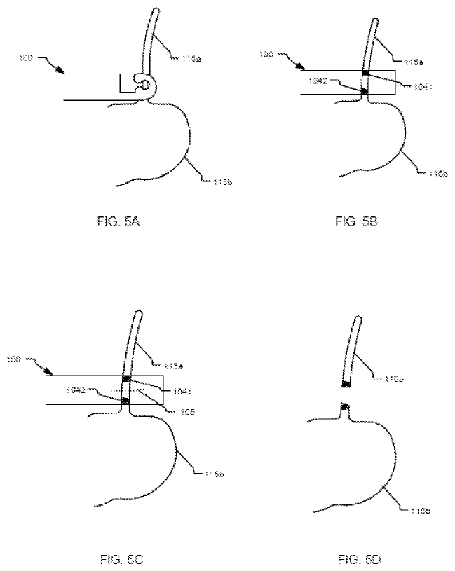

FIG. 2 illustrates exemplary perspective views of a proposed surgical instrument 100 with at least one clamping structure 104, in accordance with an embodiment of the present disclosure. As illustrated in FIG. 2, the at least one clamping structure 104 may include, a first set of pushing members 104a1 and a second set of pushing members 104a2. The first set of pushing members 104a1 and a second set of pushing members 104a2 may be coupled to the levers 107a and 107b, respectively. The at least one clamping structure 104 may include a first clamping structure 1041 and a second clamping structure 1042 (shown in FIG. 5C). The first and the second clamping structures may be configured to position adjacently, may also be referred to as proximal clamping structure and distal clamping structure, respectively. As shown in FIG. 2, at least one winding member may be coupled to the first sets of pushing members 104a1. In case of the first clamping structure 1041 and the second clamping structure 1042, the winding member 104c may be coupled to the first set of pushing members of the first and second clamping structures 1041 and 1042. As described above, the clamping may also include a set of constrictors 104b.

In an embodiment, the clamping structure 104 may include a set of the bobs attached directly to the first and the second set of the pushing members. As the handles moves from the open position to the closed position, the levers 107a and 107b may also be pushed. As a result, a set of bobs also pushed towards lever, which may enable the linear movement of the first and the second set of pushing members.

FIG. 3 illustrates exemplary perspective views of a proposed surgical instrument 100 with a cutting structure 105, in accordance with an embodiment of the present disclosure. As shown in FIG. 3, the instrument may include a cutting structure 105 that may include a trigger 105b, a pushing member 105c, rotating members 105a1 and 105a2 (collectively termed as rotating members 105a), and a cutting tool 105d. As the trigger switches from an extended position to the pushed position, rotating member 105a1 may rotated due to coupling between the rotating member 105a1 and the trigger 105b. The rotation of the rotating member 105a1 may enable the linear movement of the pushing member 105c that results in rotation of the rotating member 105a2. As the cutting tool is directly coupled to the rotating member 105a2, with the rotation of the rotating member 105a2, the cutting tool 105d may also rotate, as shown in FIG. 3. The rotation of the cutting tool 105d may facilitate the cutting of the tissue. As soon as the trigger is released, the cutting tool 105d may restore the previous position by rotating in any one of clockwise direction and anticlockwise direction.

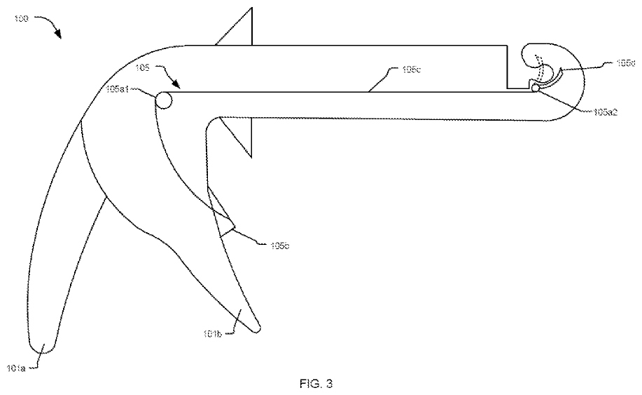

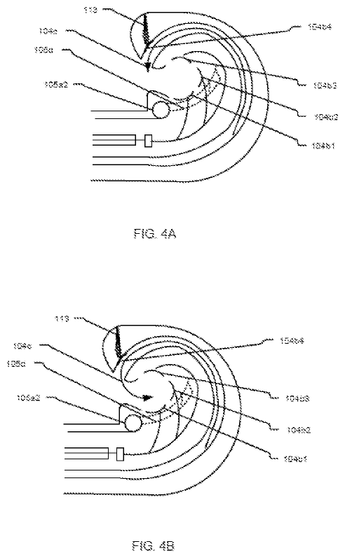

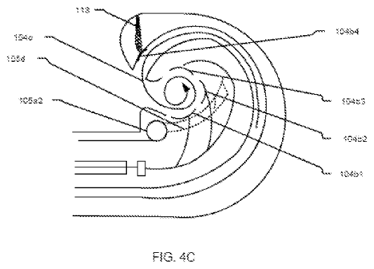

FIGS. 4A-4C illustrate exemplary perspective representation of movement of the winding member, in accordance with an embodiment of the present disclosure. When the handle 101 moves from an open position to the closed position, the first and the second sets of the pushing members 104a1 and 104a2 configured to perform their operation. Particularly, when the first set of pushing members 104a1 moves away from the lever, the set of the constrictors 104b may move from a designed position in which the set of constrictors 104b are positioned away from each other and to a contracted position in which the set of constrictors 104c are positioned closed to each other. The first set of pushing members 104a1 may push directly the constrictors 104b1, 104b2, and 104b3. In an embodiment, the instrument 100 may include a biasing member 113 that may be configured to push the constrictors 104b4 that is coupled spring 113 towards the tissue. The constrictor 104b4 guides the winder to exit from 104a2 and passes the wider to constrictor 104b1.

In another embodiment, when the second set of the pushing members 104a1 move away from the lever, the second set of the pushing members 104a2 may push the winding member 104c attached to it. The winding member 104c may be configured in a winding member box. With the movement of the second set of pushing members 104a2, the winding member 104c may move from the winding member box and exit from the gap lies between edges of the adjacent constrictors. In an preferred embodiment, the winding member 104c may exit from a gap between constrictors 104b3 and 104b4. In an embodiment, the constrictor 104b4 may guide and allow the winding member to pass through the gap between constrictors 104b3 and 104b4.

In an preferred embodiment, after passing through the gap/after exit from the constrictor, winding member may travel to 104b1 then travel 104b2 and 104b3 to form loops. The loop may be continuously formed by pressing, compressing and strangulating the tubular tissue through the entire length of the winding member. In an exemplary embodiment, the length of the winding member may be 2 cm.

FIGS. 5A-5D illustrate exemplary perspective representation of cutting operation of the proposed surgical instrument 100, in accordance with an embodiment of the present disclosure.

The cutting operation of the instrument 100 may be explained with an example of tissue as appendix. With the application of the first and the second clamping structure 1041 and 1042 configured in a hook like structure, the tissue may be hold/clamped. Then with the application of the cutting structure 105 may cut the tissue of the appendix by the cutting tool of the cutting structure 105. FIG. 5A-5C shows the cutting and clamping operation of the instrument. FIG. 5D shows appendix after performing the cutting operation.

Therefore, the present disclosure provides an improved surgical instrument that may perform clamping operation as well as the cutting operation. In particular, the clamping structure and the cutting structure enables clamping operation and cutting operation respectively. Thus, the surgical instrument may omit the need of clip applicator and the scissor. In addition, the improved surgical instrument may reduce time and minimizes the need of tissue dissection.

As used herein, and unless the context dictates otherwise, the term “coupled to” is intended to include both direct coupling (in which two elements that are coupled to each other or in contact each other) and indirect coupling (in which at least one additional element is located between the two elements). Therefore, the terms “coupled to” and “coupled with” are used synonymously. Within the context of this document terms “coupled to” and “coupled with” are also used euphemistically to mean “communicatively coupled with” over a network, where two or more devices are able to exchange data with each other over the network, possibly via one or more intermediary device.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

While some embodiments of the present disclosure have been illustrated and described, those are completely exemplary in nature. The disclosure is not limited to the embodiments as elaborated herein only and it would be apparent to those skilled in the art that numerous modifications besides those already described are possible without departing from the inventive concepts herein. All such modifications, changes, variations, substitutions, and equivalents are completely within the scope of the present disclosure. The inventive subject matter, therefore, is not to be restricted except in the spirit of the appended claims.

It should be apparent to those skilled in the art that many more modifications besides those already described are possible without departing from the inventive concepts herein. The inventive subject matter, therefore, is not to be restricted except in the spirit of the appended claims. The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

In the description of the present specification, reference to the term “one embodiment,” “an embodiments”, “an example”, “an instance”, or “some examples” and the description is meant in connection with the embodiment or example described The particular feature, structure, material, or characteristic included in the present invention, at least one embodiment or example. In the present specification, the term of the above schematic representation is not necessarily for the same embodiment or example. Furthermore, the particular features structures, materials, or characteristics described in any one or more embodiments or examples in proper manner. Moreover, those skilled in the art can be described in the specification of different embodiments or examples are joined and combinations thereof.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

### Advantages of the Invention

The present disclosure provides an improved surgical instrument that is capable of performing operation of clip applicator as well as scissor.

The present disclosure provides a surgical instrument that reduces total time spent in the surgery.

The present disclosure provides an surgical instrument that can be easily operated because of its compact size in minimal access surgeries like laparoscopic and robotic surgeries, where there will be limited operating space.

The present disclosure provides an surgical instrument that can be easily operated without damaging or disturbing the adjacent structures.

The present disclosure provides an improved surgical instrument that minimizes the need of tissue dissection.

The present disclosure provides a tubular structure ligator that may be helpful for both patients as well as for surgeons.

I claim:

1. A surgical instrument, the instrument comprising:
a handle configured to move between an open position and a closed position;
at least one clamping structure coupled to the handle, the at least one clamping structure comprising:
a first set of pushing members operatively coupled to the handle, wherein movement of the handle from open position to the closed position facilitates the movement of pushing members into longitudinal direction;
a set of constrictors coupled to the set of the pushing members such that movement of the set of the pushing members enables movement of the set of constrictors from a designed position in which the set of constrictors are positioned away from each other and to a contracted position in which the set of constrictors are positioned closed to each other,
wherein movement of the set of constrictors into contracted position facilitate clamping of a tissue.

2. The surgical instrument as claimed in claim 1, wherein the instrument comprises a cutting structure that comprises:
a trigger configured with the handle and to switch between an extended position and a pushed position;
a pushing member configured with the trigger such that switching of the trigger from the extended position to the pushed position enables linear movement of the pushing member; and
a cutting tool coupled with the pushing member such that the movement of the pushing member moves the cutting tool to cut the tissue.

3. The surgical instrument as claimed in claim 2, wherein the cutting structure comprises two rotating members, the two rotating members being movably configured on opposite side of the pushing member in longitudinal direction, wherein one of the two rotating members is movably coupled to the trigger such that switching of the trigger from the extended position to the pushed position facilitates rotation of the one of the two rotating members, and wherein another one of the two rotating members is movably coupled to the cutting tool such that rotation of the another one of the two rotating members facilitates operation of the cutting tool.

4. The surgical instrument as claimed in claim 1, wherein the at least one clamping structure comprises:
a second set of pushing members coupled to the handle; and
at least one winding member movably coupled to the second set of pushing members such that movement of the second set of pushing members enables the movement of the at least one winding member,
wherein the set of constrictors are positioned with a predefined gap between adjacent edges of the corresponding constrictors, and
wherein the at least winding member is configured to pass through the gap between two adjacent constrictors.

5. The surgical instrument as claimed in claim 1, wherein the instrument comprises a set of tubular members having hollow structure, each of the tubular members comprising each of the first and second sets of the pushing members.

6. The surgical instrument as claimed in claim 1, wherein each of the tubular members comprises a bob coupled to the pushing member of the corresponding tubular member.

7. The surgical instrument as claimed in claim 2, wherein the at least one clamping structure comprises a first clamping structure and a second clamping structure, wherein the first clamping structure is arranged with the second clamping structure, and wherein the cutting structure is configured between the first clamping structure and the second clamping structure.

8. The surgical instrument as claimed in claim 1, wherein the surgical instrument comprises a first lever coupled with the first clamping structure and a second lever coupled with the second clamping structure.

9. The surgical instrument as claimed in claim 1, wherein the set of the constrictors are positioned in a curved shape.
